(19) United States
(12) Patent Application Publication
(10) Pub. No.: US 2023/0000407 A1
(43) Pub. Date: Jan. 5, 2023

(54) Instrument Advancement Device Having a Septum

(71) Applicant: Becton, Dickinson and Company, Franklin Lakes, NJ (US)

(72) Inventors: Yiping Ma, Layton, UT (US); Jonathan Karl Burkholz, Salt Lake City, UT (US); Weston F. Harding, Lehi, UT (US); Megan Scherich, Salt Lake City, UT (US); John Lackey, West Valley City, UT (US); Erica E. Neumann, Salt Lake City, UT (US)

(21) Appl. No.: 17/852,499

(22) Filed: Jun. 29, 2022

Related U.S. Application Data
(60) Provisional application No. 63/218,069, filed on Jul. 2, 2021.

(51) Int. Cl. A61B 5/15; A61M 25/06

(52) U.S. Cl. CPC A61B 5/150992; A61M 25/0606; A61M 25/0631

(57) ABSTRACT

An instrument advancement device may include a housing, which may include a slot. The instrument advancement device may be configured to couple to a catheter assembly. The instrument advancement device may include an advancement element, which may extend through the slot and may be configured to move linearly along the slot between a retracted position and an advanced position. In response to movement of the advancement element from a retracted position to an advanced position, an instrument of the instrument advancement device may be advanced beyond a distal end of the housing and may be configured to open a path through an occlusion in the catheter assembly or vasculature. The instrument advancement device may include a septum that includes a shape and/or a lube to reduce septum resistance and buckling of the instrument during advancement of the instrument.

### CROSS-REFERENCE TO RELATED APPLICATION

The present application claims priority to U.S. Provisional Application Ser. No. 63/218,069, entitled “Instrument Advancement Device Having a Septum”, filed Jul. 2, 2021, the entire disclosure of which is hereby incorporated by reference in its entirety.

### BACKGROUND OF THE INVENTION

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. A catheter assembly may include a catheter adapter, the catheter extending distally from the catheter adapter, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Infusion and blood withdrawal using the catheter may be difficult for several reasons, particularly when an indwelling time of the catheter increases. A fibrin sheath or thrombus may form on an internal surface of the catheter assembly, an external surface of the catheter assembly, or within the vasculature near the distal tip of the catheter. The fibrin sheath or thrombus may block or narrow a fluid pathway through the catheter, which may impair infusion and/or collection of a high-quality blood sample.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure relates generally to vascular access devices and related systems and methods. In particular, the present disclosure relates to an instrument advancement device that includes a septum to facilitate advancement of an instrument of the instrument advancement device. In some embodiments, the septum may be configured to reduce or eliminate buckling of the instrument during advancement of the instrument. In some embodiments, the septum may facilitate sealing of the septum around the instrument during advancement of the instrument.

In some embodiments, the instrument advancement device may be configured to advance the instrument into a catheter assembly. In some embodiments, the instrument may be advanced through a catheter of the catheter assembly to push past any occlusions in the catheter or vasculature (e.g., thrombus or fibrin sheath at a tip of the catheter, vein collapse, or valves) to create a clear pathway for fluid flow. In some embodiments, the instrument may reduce or remove occlusions, improving patency of the catheter for medication and fluid delivery, as well as blood acquisition, during a dwell time of the catheter. In some embodiments, the catheter that may be inserted into vasculature of the patient prior to advancing the instrument in the catheter assembly and may remain in the vasculature when the instrument is advanced via the instrument advancement device. In some embodiments, the catheter may include a peripherally-inserted central catheter, a midline catheter, or a peripheral intravenous catheter.

In some embodiments, the instrument advancement device may include a housing, which may include a proximal end, a distal end, and a slot. In some embodiments, the instrument advancement device may include an advancement element, which may extend through the slot and may be configured to move linearly along the slot between a retracted position and an advanced position. In some embodiments, in response to the instrument advancement device being in the retracted position, an entirety of the instrument may be disposed within the housing. In some embodiments, a septum may be disposed within the housing. In some embodiments, the instrument may include a first end and a second end. In some embodiments, in response to movement of the advancement element from the retracted position to the advanced position, the instrument may move through the septum and the second end of the instrument may be advanced beyond the distal end of the housing.

In some embodiments, a distal face of the septum may be flat, which may reduce a compression force to seal the septum within the housing. In some embodiments, the distal face of the septum may face a fluid path within the housing, and blood within the fluid path may help seal an interface between the septum and the instrument extending therethrough.

In some embodiments, a distal face of the septum may be bulging and a proximal face of the septum may be bulging. In further detail, in some embodiments, a bulge of the distal face and a bulge of the proximal face may be aligned and/or aligned with a central axis of the septum. In some embodiments, the bulge of the distal face and the bulge of the proximal face may be aligned with a central axis of the fluid path of the housing. In some embodiments, a maximum width of the septum may be disposed at the bulge of the distal face and the bulge of the proximal face. In some embodiments, the pressure of blood within the fluid path of the housing against the bulge of the distal face may facilitate sealing of the septum during advancement of the instrument. Moreover, in some embodiments, a pressure differential resulting from a blood collection device, such as, for example, a BD VACUTAINER® blood collection tube available from Becton Dickinson & Company of Franklin Lakes, N.J., may facilitate sealing of the septum during blood draw.

In some embodiments, the distal face of the septum may be concave and the proximal face of the septum may be concave, which may ensure the pressure of blood within the fluid path or the pressure differential resulting from the blood collection device is over the interface between the septum and the instrument.

In some embodiments, the housing may include a support wall. In some embodiments, the support wall may include an opening configured to receive the instrument therethrough. In some embodiments, the distal face of the septum may be in contact with the support wall. In some embodiments, the support wall may extend perpendicular to a longitudinal axis of the housing. In some embodiments, a center of the septum is aligned with the opening of the support wall. In some embodiments, the septum may include a duckbill valve.

In some embodiments, the instrument advancement device may include another septum, which may be disposed within the housing. In some embodiments, the septum may be in contact with a proximal side of the support wall and/or the other septum may be in contact with a distal side of the support wall. In some embodiments, a portion of the septum aligned with opening may include a proximally-facing pocket configured to receive the instrument therethrough. In some embodiments, the instrument advancement device may include a lube disposed within the proximally-facing pocket.

In some embodiments, the instrument advancement device may include a wedge. In some embodiments, the wedge may include a distal end and an outwardly-extending mouth portion opposite the distal end. In some embodiments, the distal end of the wedge may be secured within the opening of the support wall. In some embodiments, a proximal face of the septum may be in contact with a distal side of the support wall. In some embodiments, the outwardly-extending mouth portion may include a lube therein.

In some embodiments, the lube may be disposed within the housing and configured to facilitate advancement of the instrument through the septum. In some embodiments, another septum may be disposed within the housing, and the lube may fill a volume between the septum and the other septum.

In some embodiments, the septum may include a proximally-facing pocket, and a distal end of the wedge may be disposed within the proximally-facing pocket of the septum. In some embodiments, an outwardly-angled mouth of the wedge may include the lube therein. In some embodiments, the outwardly-angled mouth of the wedge may be disposed proximal to the septum.

In some embodiments, a distal face of the septum may be flat and a proximal face of the septum may include a pocket configured to receive the instrument therethrough. In some embodiments, the lube may be disposed within the pocket. In some embodiments, the septum may include a cavity enclosed by a body of the septum, and the lube is disposed within the cavity enclosed by the body of the septum.

In some embodiments, the distal face of the septum may include a distally-facing pocket, and the instrument advancement device may include a film or another septum. In some embodiments, the lube may be sealed within the distally-facing pocket by the film or the other septum. In some embodiments, the lube may fill a volume between the septum and the film. In some embodiments, in response to movement of the advancement element from the retracted position to the advanced position, the instrument may be configured to puncture the film or the other septum to release the lube.

In some embodiments, the film may be proximal to the septum. In some embodiments, the lube may fill a volume between the septum and the film. In some embodiments, in response to movement of the advancement element from the retracted position to the advanced position, the instrument may be configured to puncture the film to release the lube.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective view of an example instrument advancement device, according to some embodiments;

FIG. 1B is a cross-sectional view of the instrument advancement device;

FIG. 1C is a cross-sectional view of the instrument advancement device along the line 1C-1C of FIG. 1A, according to some embodiments;

FIG. 1D is an enlarged view of a portion of FIG. 1C, according to some embodiments;

FIG. 1E is a cross-sectional view of the instrument advancement device along the line 1E-1E of FIG. 1A, according to some embodiments;

FIG. 2A is a cross-sectional view of the instrument advancement device, according to some embodiments;

FIG. 2B is an enlarged view of a portion of the cross-sectional view of FIG. 2A;

FIG. 2C is an enlarged view of a portion of the cross-sectional view of FIG. 2A, illustrating an example lube, according to some embodiments;

FIG. 3A is a cross-sectional view of the instrument advancement device, according to some embodiments;

FIG. 3B is an enlarged view of a portion of the cross-sectional view of FIG. 3A;

FIG. 4A is a cross-sectional view of the instrument advancement device, according to some embodiments;

FIG. 4B is an enlarged view of a portion of the cross-sectional view of FIG. 4A;

FIG. 5A is a cross-sectional view of the instrument advancement device, according to some embodiments;

FIG. 5B is an enlarged view of a portion of the cross-sectional view of FIG. 5A;

FIG. 6A is a cross-sectional view of the instrument advancement device, according to some embodiments;

FIG. 6B is an enlarged view of a portion of the cross-sectional view of FIG. 6A;

FIG. 7A is a cross-sectional view of the instrument advancement device, according to some embodiments;

FIG. 7B is an enlarged view of a portion of the cross-sectional view of FIG. 7A;

FIG. 8A is a cross-sectional view of the instrument advancement device, according to some embodiments;

FIG. 8B is an enlarged view of a portion of the cross-sectional view of FIG. 8A;

FIG. 9A is a cross-sectional view of the instrument advancement device, according to some embodiments;

FIG. 9B is an enlarged view of a portion of the cross-sectional view of FIG. 9A;

FIG. 10A is a cross-sectional view of the instrument advancement device, according to some embodiments;

FIG. 10B is an enlarged view of a portion of the cross-sectional view of FIG. 10A;

FIG. 10C is an enlarged view of a portion of the cross-sectional view of the instrument advancement device, according to some embodiments;

FIG. 11A is a cross-sectional view of the instrument advancement device, according to some embodiments;

FIG. 11B is an enlarged view of a portion of the cross-sectional view of FIG. 11A;

FIG. 11C is an enlarged view of a portion of the cross-sectional view of the instrument advancement device, according to some embodiments;

FIG. 11D is an enlarged view of a portion of the cross-sectional view of the instrument advancement device, according to some embodiments;

FIG. 12A is a cross-sectional view of the instrument advancement device, according to some embodiments;

FIG. 12B is an enlarged view of a portion of the cross-sectional view of FIG. 12A;

FIG. 13A is an upper perspective view of another instrument advancement device, according to some embodiments;

FIG. 13B is a cross-sectional view of an example distal end of the other instrument advancement device, according to some embodiments;

FIG. 13C is an upper perspective view of a distal end of an example extension tube of the other instrument advancement device, according to some embodiments; and

FIG. 13D is a cross-sectional view of an example housing of the other instrument advancement device, according to some embodiments.

### DETAILED DESCRIPTION

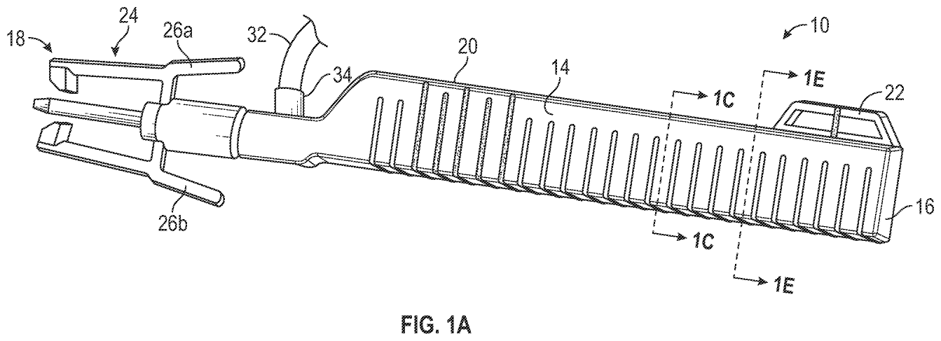

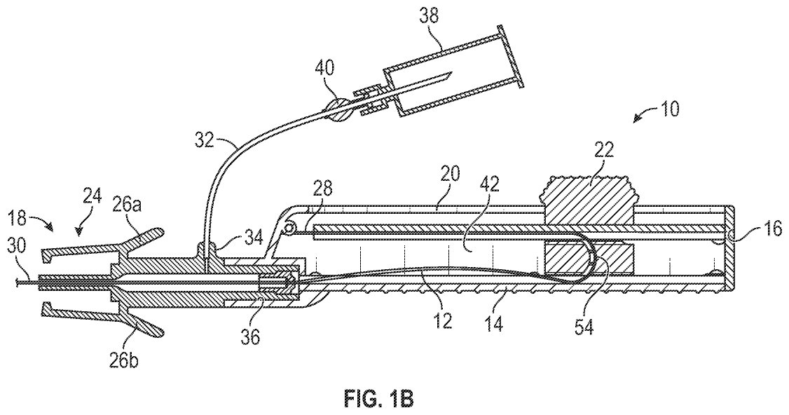

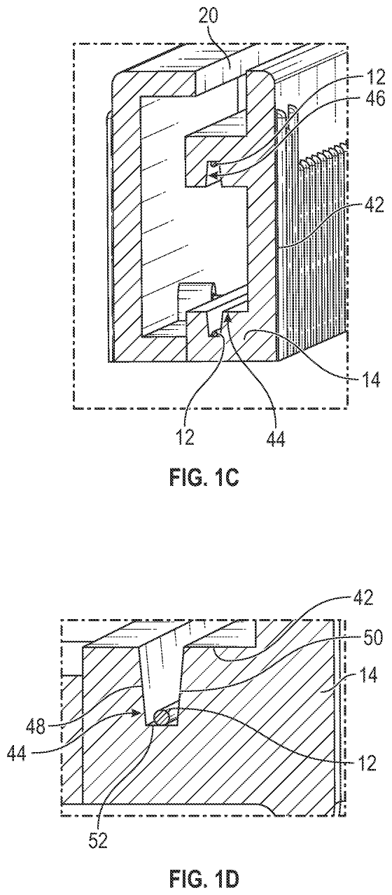

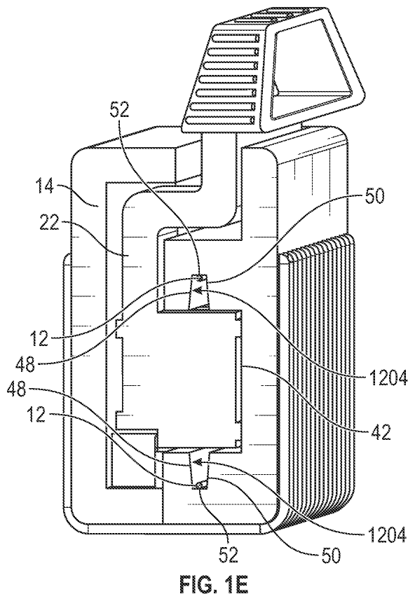

Referring now to FIGS. 1A-1E, in some embodiments, an instrument advancement device 10 may be configured to deliver an instrument 12 into and/or through a catheter of a catheter assembly. In some embodiments, the instrument 12 may be advanced through the catheter to push past any occlusions in the catheter or vasculature (e.g., thrombus or fibrin sheath at a tip of the catheter, vein collapse, valves, etc.) to create a clear pathway for fluid flow. In some embodiments, the instrument 12 may reduce or remove occlusions, improving patency of the catheter for medication and fluid delivery, as well as blood acquisition, during a dwell time of the catheter.

In some embodiments, the instrument 12 may include a guidewire, a probe, a guidewire or a probe with one or more sensors, or another suitable instrument. In some embodiments, the sensors may be used for patient or device monitoring and may include sensors measuring pressure, temperature, pH, blood chemistry, oxygen saturation, flow rate, or another physiological property. In some embodiments, the catheter may include a peripheral intravenous (IV) catheter, a peripherally-inserted central catheter, or a midline catheter. In some embodiments, the catheter through which the instrument 12 may be delivered may have been previously inserted into vasculature of a patient and may be dwelling within the vasculature when the instrument 12 is advanced through the catheter.

In some embodiments, the instrument 12 may be disposed within a housing 14, which may be configured to protect the instrument 12 from damage and/or contamination from a surrounding external environment. In some embodiments, the housing 14 may be rigid or semi-rigid. In some embodiments, the housing 14 may be made of one or more of stainless steel, aluminum, polycarbonate, metal, ceramic, plastic, and another suitable material. In some embodiments, the housing 14 may include a proximal end 16, a distal end 18, and a slot 20. In some embodiments, the slot 20 may extend parallel to a longitudinal axis of the housing 14.

In some embodiments, the instrument advancement device 10 may include an advancement element 22, which may extend through the slot 20 and may be configured to move linearly along the slot 20 between a retracted position illustrated, for example, in FIG. 1A, and an advanced position. In some embodiments, the clinician may pinch or grasp the advancement element 22 to move the advancement element 22 between the retracted position and the advanced position.

In some embodiments, the distal end 18 of the housing 14 may include a connector 24. In some embodiments, the connector 24 may include opposing lever arms 26a,26b. In some embodiments, distal ends of the opposing lever arms 26a,26b may be configured to move apart from each other in response to pressure applied to proximal ends of the opposing lever arms 26a,26b. In some embodiments, in response to removal of the pressure applied to the proximal ends of the opposing lever arms 26a,26b, the distal ends may move closer to each other and clasp a portion of the catheter assembly, such as a needleless connector, another connector, or a proximal end of a catheter adapter, for example. In some embodiments, the connector 24 may include a blunt cannula or male luer configured to insert into the portion of the catheter assembly.

In some embodiments, the connector 24 may include any suitable connector. For example, the connector 24 may include a threaded male luer, a slip male luer, a threaded male luer with a spin lock, a threaded male luer with a removable blunt cannula snap connection, a slip male luer with a removable blunt cannula snap connection, or another suitable connector. In some embodiments, the connector 24 may include one or more bond pockets, which may each be configured to receive an extension tube. In some embodiments, the connector 24 may be monolithically formed as a single unit with a body of the housing 14 that includes the slot 20.

In some embodiments, the instrument 12 may include a first end 28 and a second end 30. In some embodiments, movement of the advancement element 22 from the retracted position to the advanced position may cause the second end 30 of the instrument 12 to be advanced beyond the distal end 18 of the housing 14. In some embodiments, moving the advancement element 22 to the advanced position may introduce the instrument 12 into the catheter assembly and/or through the catheter. In some embodiments, in response to the instrument 12 being introduced into the catheter assembly and/or through the catheter, the instrument 12 may access a fluid pathway of the catheter assembly and/or the vasculature of a patient.

In some embodiments, the catheter of the catheter assembly with significant dwelling time within the vasculature may be susceptible to narrowing, collapse, kinking, blockage by debris (e.g., fibrin or platelet clots), and adhering of a tip of the catheter to the vasculature. Thus, blood withdrawal using the catheter may be difficult. In some embodiments, the instrument 12 may have a diameter less than a diameter of the catheter of the catheter assembly to provide access to the vasculature of the patient without any additional needle sticks. In some embodiments, the instrument 12 may clear the pathway for collecting a blood sample. Thus, in some embodiments, the instrument advancement device 10 may be used for needle-free blood collection and/or fluid infusion.

In some embodiments, an extension tube 32 may be coupled to the instrument advancement device 10, and the extension tube 32 may be used for blood collection and/or fluid infusion. In some embodiments, the extension tube 32 may extend from a port 34 of the housing 14 or another portion of the housing 14.

In some embodiments, a septum 36 may be within the housing 14 to enable the instrument 12 to advance and/or retract while maintaining a closed fluid path. In some embodiments, the instrument 12 may be configured to extend through the septum 36. In some embodiments, the septum 36 may be disposed proximal to the port 34 and/or distal to the advancement element 22 in the advanced position. In some embodiments, the septum 36 may include silicone, rubber, an elastomer, or another suitable material. In some embodiments, the septum 36 may include a slit to accommodate the instrument 12 therethrough.

In some embodiments, a proximal end of the extension tube 32 may be coupled to a blood collection device 38. For example, the proximal end of the extension tube 32 may be integrated with a connector 40, which may be coupled to the blood collection device 38. In some embodiments, a needleless connector may be disposed between the connector 40 and the blood collection device 38. In some embodiments, the connector 40 and/or the port 34 may be coupled to an IV line or another fluidic connection.

In some embodiments, an inner surface 42 of the housing 14 may include one or more grooves. For example, the inner surface 42 may include a first groove 44 and/or a second groove 46. In some embodiments, the first groove 44 and/or the second groove 46 may be disposed within the housing 14 between the proximal end 16 and the distal end 18. In some embodiments, the instrument 12 may be disposed within the first groove 44 and/or the second groove 46. In some embodiments, the first groove 44 and/or the second groove 46 may include a side wall 48, another side wall 50 opposite the side wall, and a bottom 52 extending between the side wall 48 and the other side wall 50. In some embodiments, the first groove 44 and/or the second groove 46 may be open opposite the bottom 52. In some embodiments, the first groove 44 and/or the second groove 46 may be linear and/or configured to guide the instrument 12 as the instrument 12 is advanced distally and/or retracted proximally.

In some embodiments, the advancement element 22 may include an arc-shaped channel 54, which may be U-shaped. In some embodiments, the instrument 12 may extend and move through the arc-shaped channel 54. In some embodiments, the first end 28 of the instrument 12 may be fixed. In some embodiments, the first end 28 of the instrument may be fixed within the housing 14. In some embodiments, in response to movement of the advancement element 22 a first distance, the second end of the instrument 12 may be configured to advance distally a second distance. In some embodiments, the second distance may be twice the first distance. In some embodiments, the second distance may be more than twice the first distance. In these and other embodiments, the instrument 12 may extend through multiple U-shapes or other arc-shapes.

In some embodiments, the instrument advancement device 10 may include any suitable instrument advancement device that includes an advancement element configured to move linearly along a slot of a housing to advance and/or retract the instrument 12. For example, the first end 28 of the instrument 12 may be fixed to the advancement element 22, and in response to the advancement element 22 moving a distance, the second end 30 of the instrument 12 may move a distance equal to the distance such that advancement ratio between the instrument 12 and the advancement element 22 is 1:1. In some embodiments, because the first groove 44 and/or the second groove 46 are open opposite the bottom 52, the instrument 12 may tend to buckle in response to the advancement element 22 being advanced distally, as illustrated, for example, in FIG. 1B.

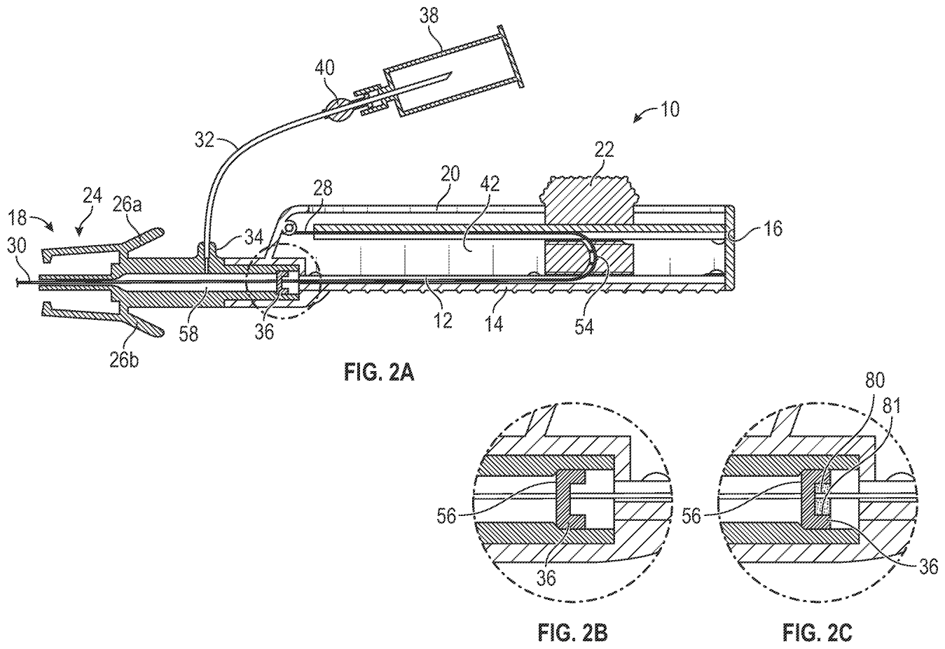

Referring now to FIG. 2A, in some embodiments, the septum 36 disposed within the housing 14 may be configured to reduce or eliminate buckling of the instrument 12 when the instrument 12 is advanced distally. In some embodiments, in response to movement of the advancement element 22 from the retracted position to the advanced position, the instrument 12 may move through the septum 36 and the second end 30 of the instrument 12 may be advanced beyond the distal end 18 of the housing 14. In some embodiments, in response to the instrument advancement device 10 being in the retracted position, an entirety of the instrument 12 may be disposed within the housing 14 and/or the advancement element 22 may be disposed at a proximal end of the slot 20. FIG. 2A illustrates the advancement element 22 moving between the retracted position and the advanced position, according to some embodiments. In some embodiments, when the advancement element 22 is in the advanced position, the advancement element 22 may be disposed at a distal end of the slot 20 and the instrument 12 may be fully advanced in a distal direction.

As illustrated in FIGS. 2A-2B, in some embodiments, a distal face 56 of the septum 36 may be flat, which may reduce a compression force to seal the septum 36 within the housing 14. In some embodiments, the distal face 56 of the septum 36 may face a fluid path 58 within the housing 14, and blood within the fluid path 58 may help seal an interface between the septum 36 and the instrument 12 extending therethrough. In some embodiments, the blood may flow proximally through the catheter, catheter adapter, and the catheter assembly into the fluid path 58 disposed within the housing 14. In some embodiments, the septum 36 may prevent the blood from flowing proximal to the septum 36 within the housing 14, thereby preventing the blood from exiting the slot 20 and exposing a user.

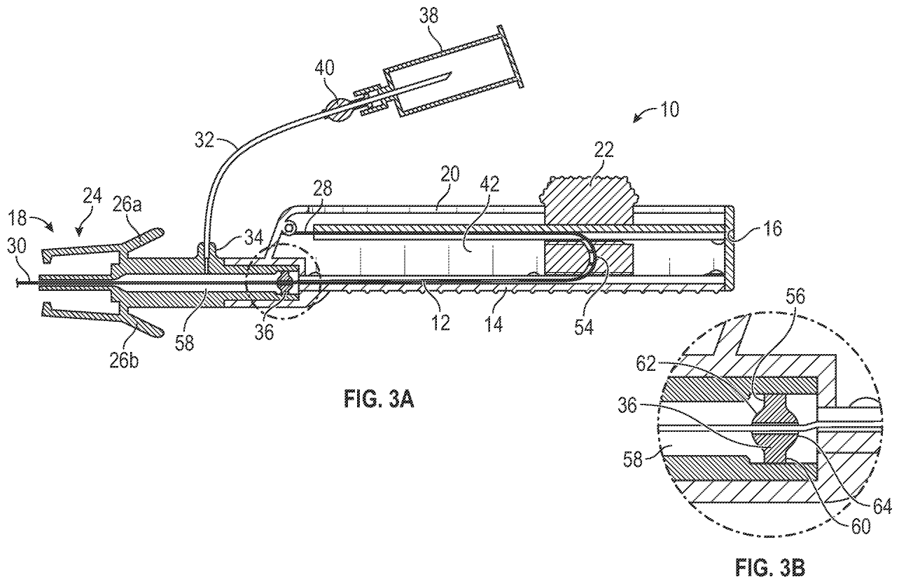

Referring now to FIGS. 3A-3B, in some embodiments, the distal face 56 of the septum 36 may be bulging and a proximal face 60 of the septum 36 may be bulging. In further detail, in some embodiments, a bulge 62 of the distal face 56 and a bulge 64 of the proximal face 60 may be aligned and/or aligned with a central axis of the septum 36. In some embodiments, the bulge 62 of the distal face 56 and the bulge 64 of the proximal face 60 may be aligned with a central axis of the fluid path 58 of the housing 14. In some embodiments, the bulge 62 and the bulge 64 may be equally sized. In some embodiments, a maximum width of the septum 36 may be disposed at the bulge 62 of the distal face 56 and the bulge 64 of the proximal face 60.

In some embodiments, the pressure of the blood within the fluid path 58 against the bulge 62 of the distal face 56 may facilitate sealing of the septum 36 around the instrument 12 during advancement of the instrument 12. Moreover, in some embodiments, a pressure differential resulting from a blood collection device, such as, for example, the blood collection device 38, may apply pressure to the bulge 64 of the proximal face 60 and facilitate sealing of the septum 36 around the instrument 12 during blood draw. In some embodiments, the blood collection device 38 may include a syringe, a BD VACUTAINER® blood collection tube available from Becton Dickinson & Company of Franklin Lakes, N.J., or another suitable blood collection device.

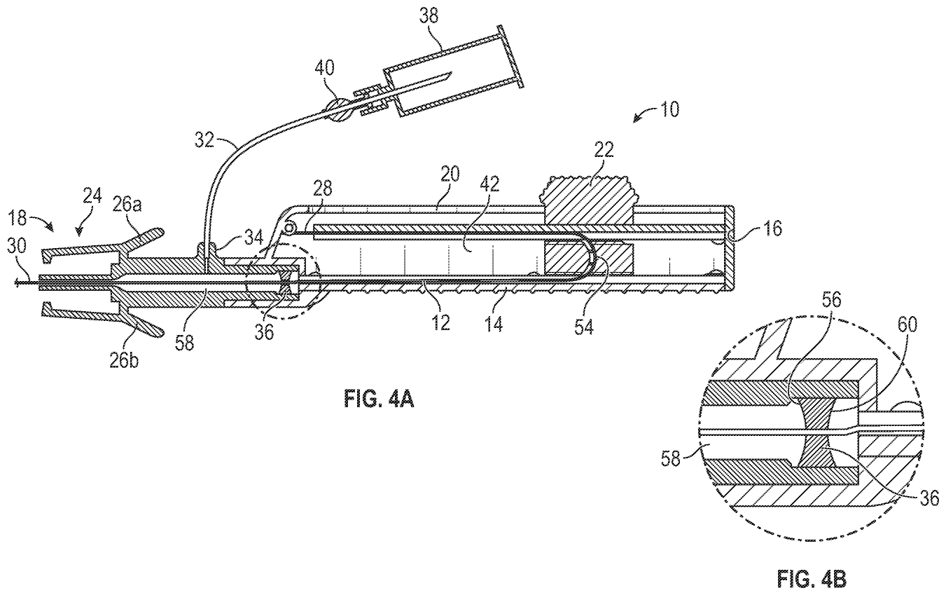

Referring now to FIGS. 4A-4B, in some embodiments, the distal face 56 of the septum 36 may be concave and the proximal face 60 of the septum 36 may be concave, which may ensure the pressure generated due to interference between the septum 36 and the housing 14 is over the interface between the septum 36 and the instrument 12.

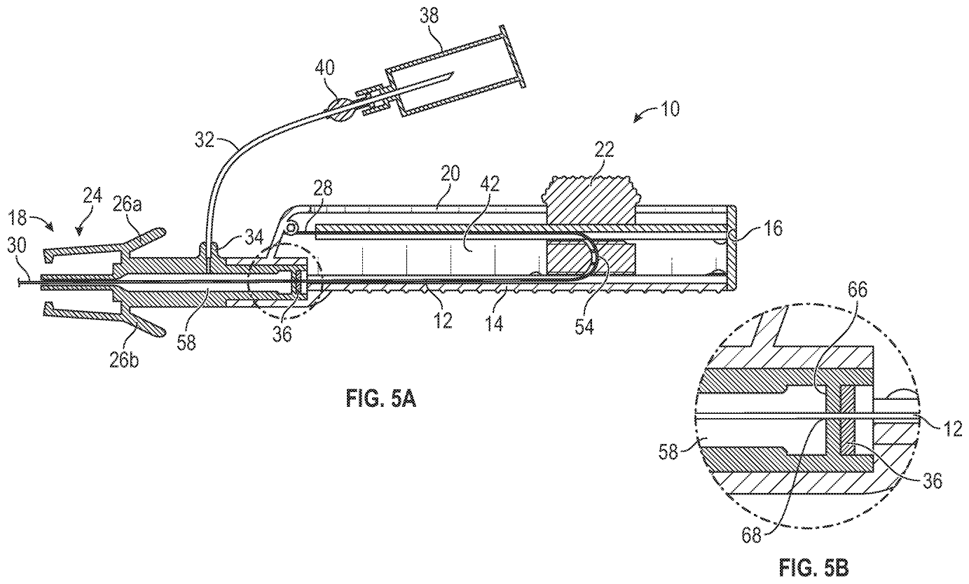

Referring now to FIGS. 5A-5B, in some embodiments, the housing 14 may include a support wall 66. In some embodiments, the support wall 66 may include an opening 68 configured to receive the instrument 12 therethrough. In some embodiments, the distal face 56 of the septum 36 may be in contact with the support wall 66. In some embodiments, the support wall 66 may extend perpendicular to a longitudinal axis of the housing 14 and/or the fluid path 58. In some embodiments, a center of the septum 36 may be aligned with the opening 68 of the support wall 66. In some embodiments, the opening 68 in combination with the support wall 66 may prevent the septum 36 from bowing and leaking when a vacuum is applied distal to the septum 36 during blood draw via the blood collection device 38.

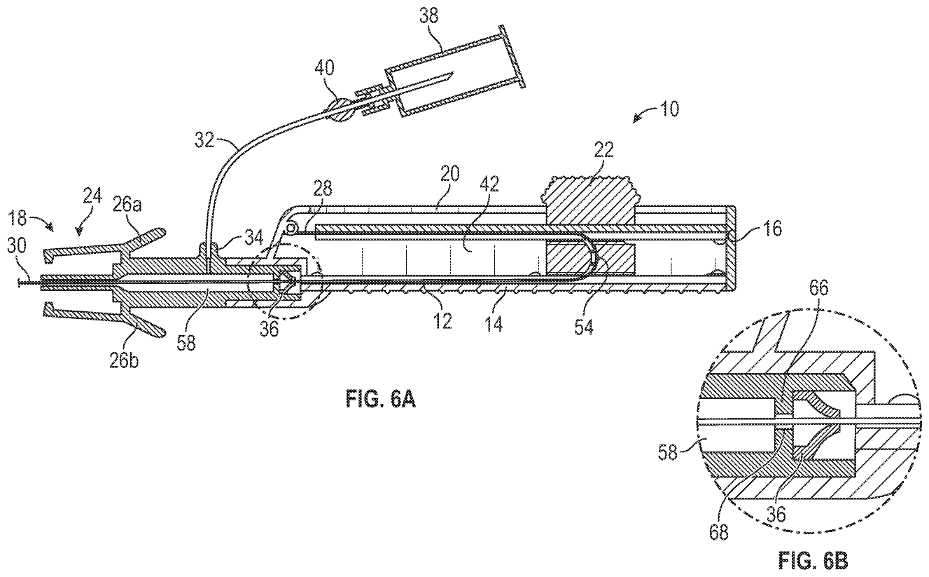

Referring now to FIGS. 6A-6B, in some embodiments, the septum 36 may include a duckbill valve. In some embodiments, the duckbill valve may help seal an interface between the septum 36 and the instrument 12 during advancement of the instrument 12 and/or during blood draw when the vacuum is applied.

In some embodiments, the housing 14 may include the support wall 66, which may include the opening 68 configured to receive the instrument 12 therethrough. In some embodiments, the distal face 56 of the septum 36 may be in contact with the support wall 66. In some embodiments, the opening 68 in combination with the support wall 66 may prevent the septum 36 from bowing and leaking when the vacuum is applied distal to the septum 36 during blood draw.

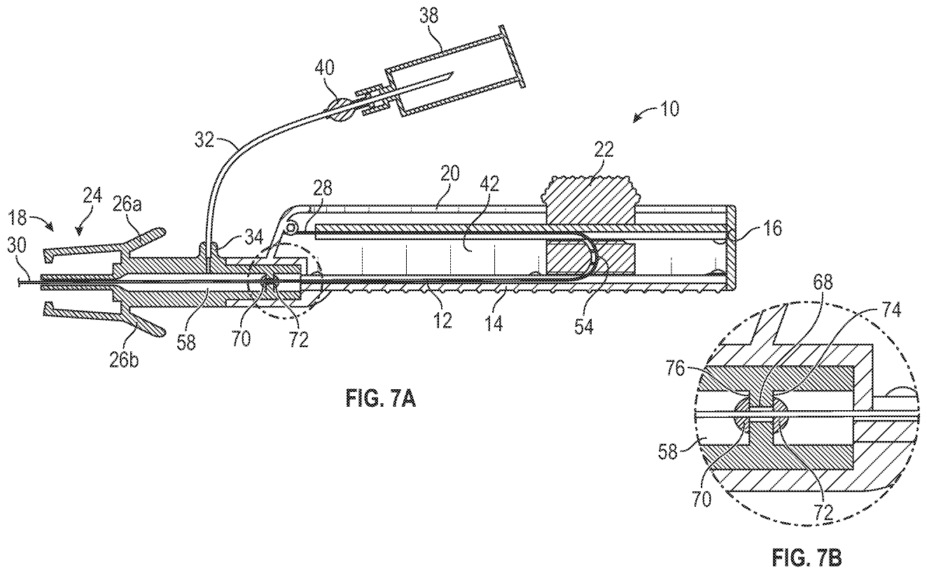

Referring now to FIGS. 7A-7B, in some embodiments, the instrument advancement device 10 may include a septum 70 and another septum 72 , which may be disposed within the housing 14. In some embodiments, the septum 70 and/or the other septum 72 may be similar or identical to the septum 36 in terms of one or more features and/or operation. In some embodiments, the other septum 72 may be in contact with a proximal side 74 of the support wall 66 and/or the septum 70 may be in contact with a distal side 76 of the support wall 66. In some embodiments, the septum 70 and the other septum 72 may help seal an interface between the septum 36 and the instrument 12 during advancement of the instrument 12 and/or during blood draw when the vacuum is applied. Moreover, in some embodiments, the opening 68 in combination with the support wall 66 may prevent the septum 70 and the other septum 72 from bowing and leaking when the vacuum is applied distal to the septum 70 during blood draw and when the blood in the fluid path 58 contacts the septum 70.

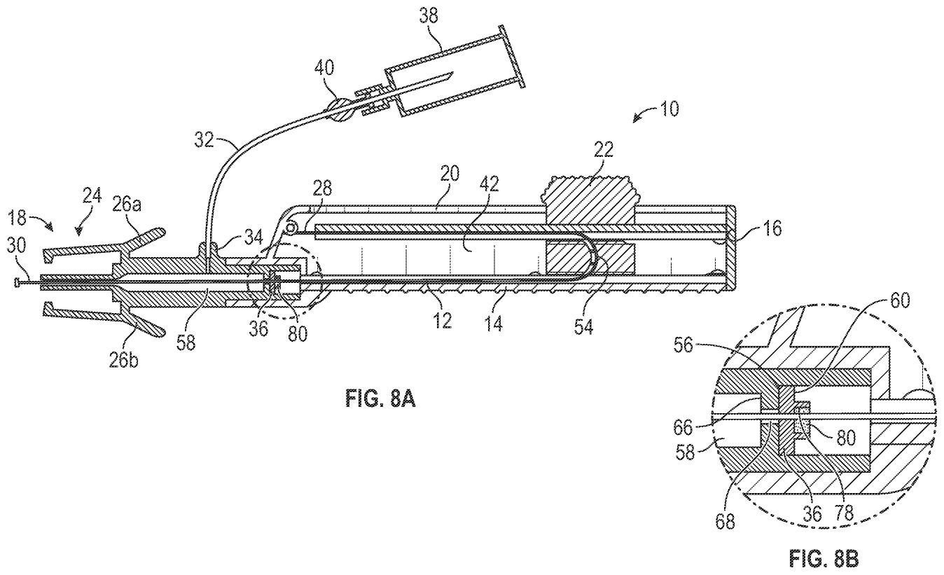

Referring now to FIGS. 8A-8B, in some embodiments, a portion of the septum 36 aligned with the opening 68 may include a proximally-facing pocket 78 configured to receive the instrument 12 therethrough. In some embodiments, the proximally-facing pocket 78 may protrude proximally from the proximal face 60. In some embodiments, the instrument advancement device 10 may include a lube 80 disposed within the proximally-facing pocket 78. In some embodiments, the lube 80 may reduce friction between the septum 36 and the instrument 12, thus reducing a likelihood of the instrument 12 buckling when the instrument 12 advances through the septum 36. In some embodiments, the lube 80 may include a high viscosity lube, which may help seal the interface between the septum 36 and the instrument 12. In some embodiments, the lube 80 may include silicone or another suitable material.

Referring back to FIG. 2C, in some embodiments, the lube 80 may be in a proximally-facing pocket 81, which may provide less migration of the lube 80 and help seal the interface between the septum 36 and the instrument 12. In these and other embodiments, the lube 80 may provide sealing to reduce air leakage through the septum 36 and reduce friction.

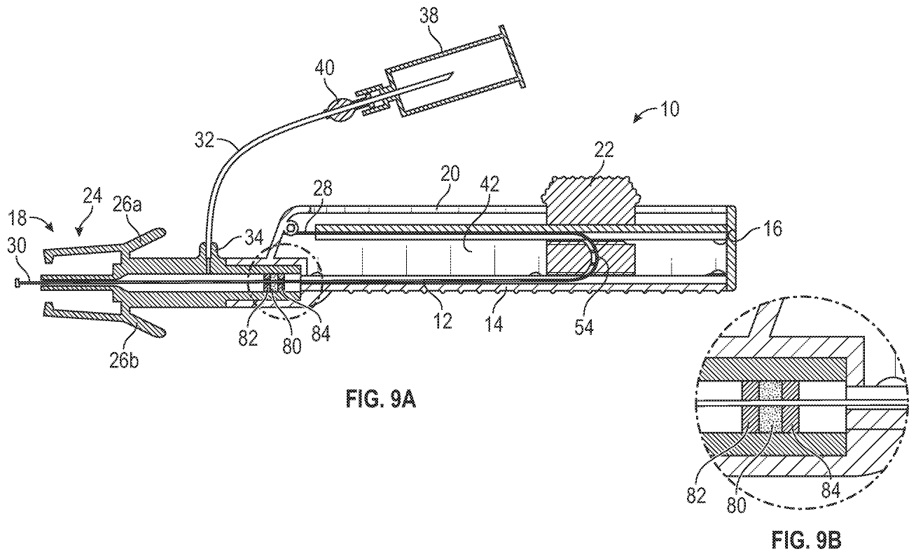

Referring now to FIGS. 9A-9B, in some embodiments, a septum 82 and another septum 84 may be disposed in the housing 14 and spaced apart by a volume. In some embodiments, the septum 82 and/or the other septum 84 may be similar or identical to the septum 36 in terms of one or more features and/or operation. In some embodiments, the lube 80 may fill the volume between the septum 82 and the other septum 84. In some embodiments, the lube 80 may reduce friction between the septum 82 and the instrument 12 and the septum 84 and the instrument 12, thus reducing a likelihood of the instrument 12 buckling when the instrument 12 advances through the septum 82 and the septum 84. In some embodiments, the lube 80 may include a high viscosity lube, which may help seal the interface between the septum 82 and the instrument 12 and the septum 84 and the instrument 12.

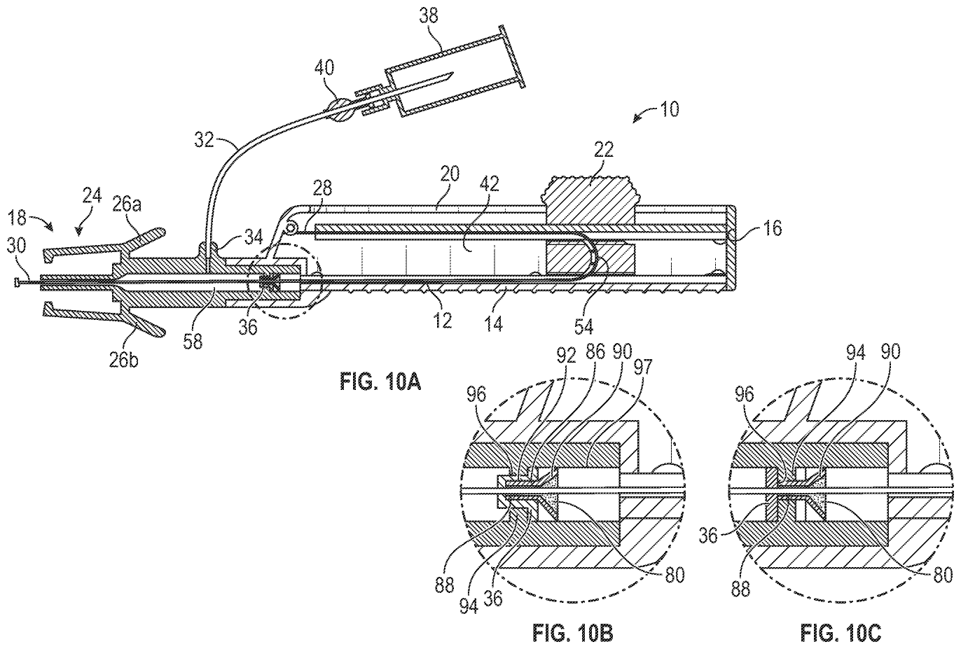

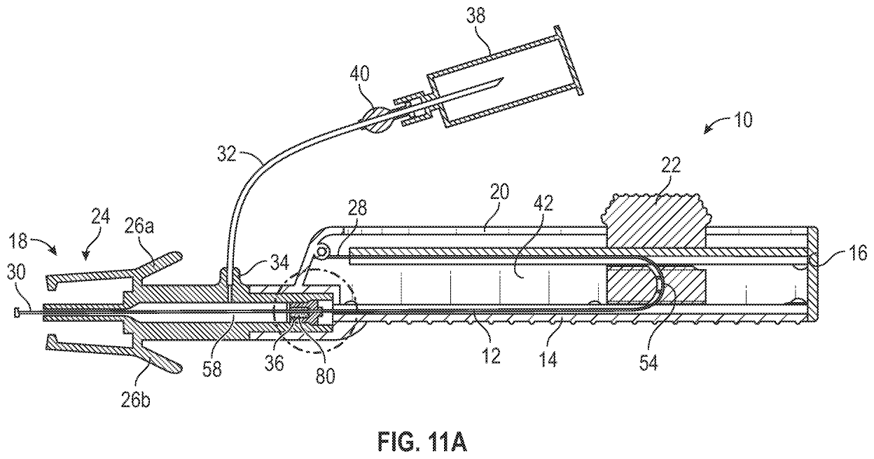

Referring now to FIGS. 10A-10B, in some embodiments, the instrument advancement device 10 may include a wedge 86. In some embodiments, the wedge 86 may include a distal end 88 and an outwardly-extending mouth portion 90 opposite the distal end 88. In some embodiments, the distal end 88 of the wedge 86 may be secured within the opening 68 of the support wall 66. In some embodiments, the outwardly-extending mouth portion 90 and/or the distal end 88 may include the lube 80 therein. In some embodiments, a shape of the outwardly-extending mouth portion 90 may facilitate securement of an increased amount of the lube 80 within the wedge 86. In some embodiments, the lube 80 may facilitate advancement of the instrument 12 through the septum 36 and sealing of the septum 36 around the instrument 12.

In some embodiments, the wedge 86 may be constructed of metal or another suitable material. In some embodiments, the septum 36 may include a proximally-facing pocket 92, and the distal end of the wedge 86 may be disposed within the proximally-facing pocket 92 of the septum 36. In some embodiments, the outwardly-extending mouth portion 90 of the wedge 86 may be disposed proximal to the septum 36.

In some embodiments, the housing 14 may include a wall 94, which may include an opening 96. In some embodiments, the distal end 88 and the septum 36 may be engaged in an interference fit with the wall 94. Additionally or alternatively, in some embodiments, the outwardly-extending mouth portion 90 may be engaged in an interference fit with an inner surface 97 of the housing 14, which may facilitate securement of the wedge 86 within the housing 14.

In some embodiments, the instrument 12 may pass through the wedge 86 and the septum 36 to advance into the fluid path 58. In some embodiments, a gap between an inner diameter of the wedge 86 and the instrument 12 can be very small and substantially or entirely sealed by the lube 80. In some embodiments, the wedge 86 and the lube 80 may provide sealing redundancy, which may allow the septum 36 to be thinner and therefore have lower drag force on the instrument 12.

Referring now to FIG. 10C, in some embodiments, the septum 36 may contact a distal side of the wall 94, which may support the septum 36. In these embodiments, the distal end 88 of the wedge 86 may be engaged in an interference fit within the opening 96, which may secure the wedge 86 within the housing 14. In some embodiments, the wedge 86 in FIGS. 10A-10B or 10C-10D may be replaced with a cannula to provide a very small clearance with the instrument 12. In some embodiments, the cannula may be constructed of metal or another suitable material. In some embodiments, the cannula may not include an outwardly-extending mouth portion 90 but may otherwise be similar to the wedge 86 in terms of one or more features and/or operation.

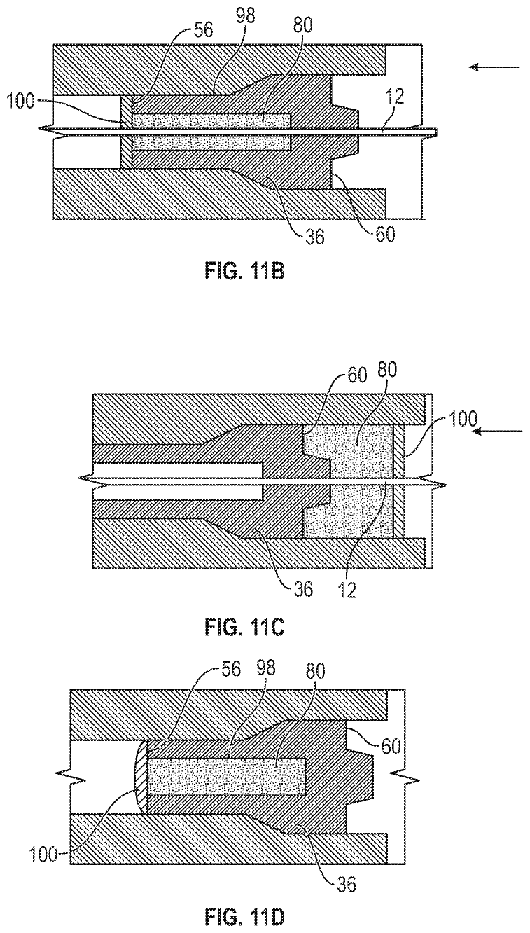

Referring now to FIGS. 11A-11B, in some embodiments, the distal face 56 of the septum 36 may include a distally-facing pocket 98, and the instrument advancement device 10 may include a barrier 100, which may include a film or another septum. In some embodiments, the lube 80 may be sealed within the distally-facing pocket 98 by the barrier 100. In some embodiments, the lube 80 may fill a volume between the septum 36 and the barrier 100. In some embodiments, in response to movement of the advancement element 22 from the retracted position to the advanced position, the instrument 12 may be configured to puncture the barrier 100 to release the lube 80.

Referring now to FIG. 11C, in some embodiments, the barrier 100 may be proximal to the septum 36. In some embodiments, the lube 80 may fill a volume between the septum 36 and the barrier 100. In some embodiments, in response to movement of the advancement element 22 from the retracted position to the advanced position, the instrument 12 may be configured to puncture the barrier to release the lube 80.

FIGS. 11A-11C illustrate the barrier 100 may be a film, according to some embodiments. Referring now to FIG. 11D, the barrier 100 may include another septum or secondary septum. In some embodiments, the other septum may be elastomeric. In some embodiments, a distal face of the other septum may be rounded.

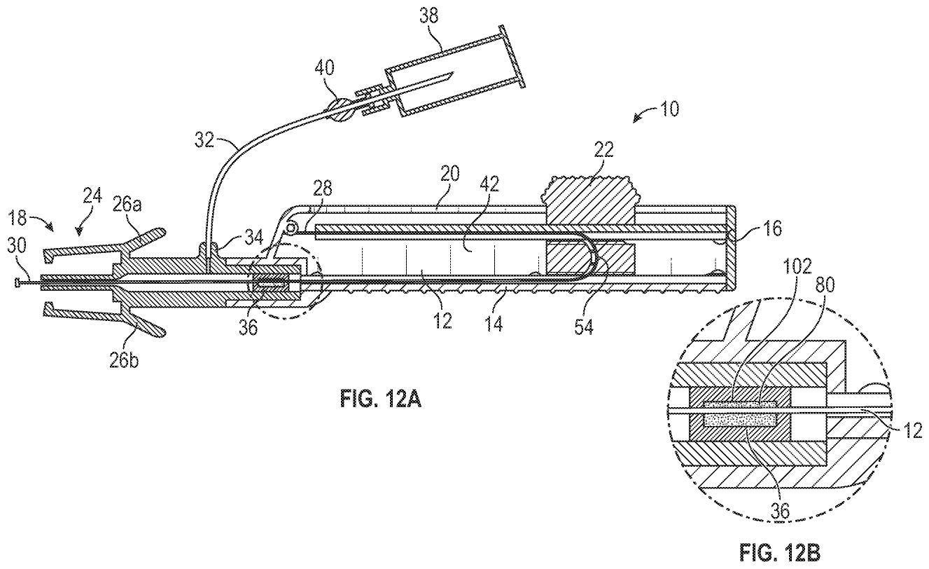

Referring now to FIGS. 12A-12B, in some embodiments, the septum 36 may include a cavity 102 enclosed by a body of the septum 36, and the lube 80 may be disposed within the cavity 102 enclosed by the body of the septum 36.

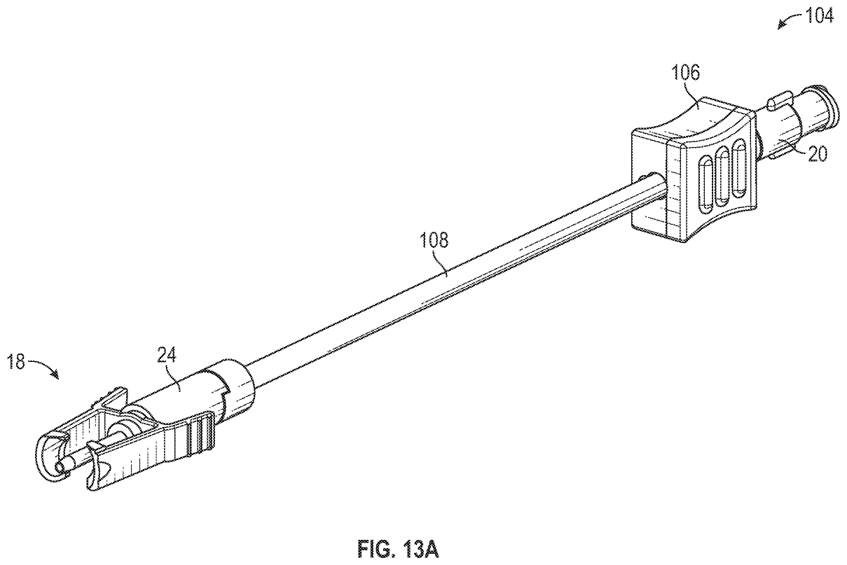

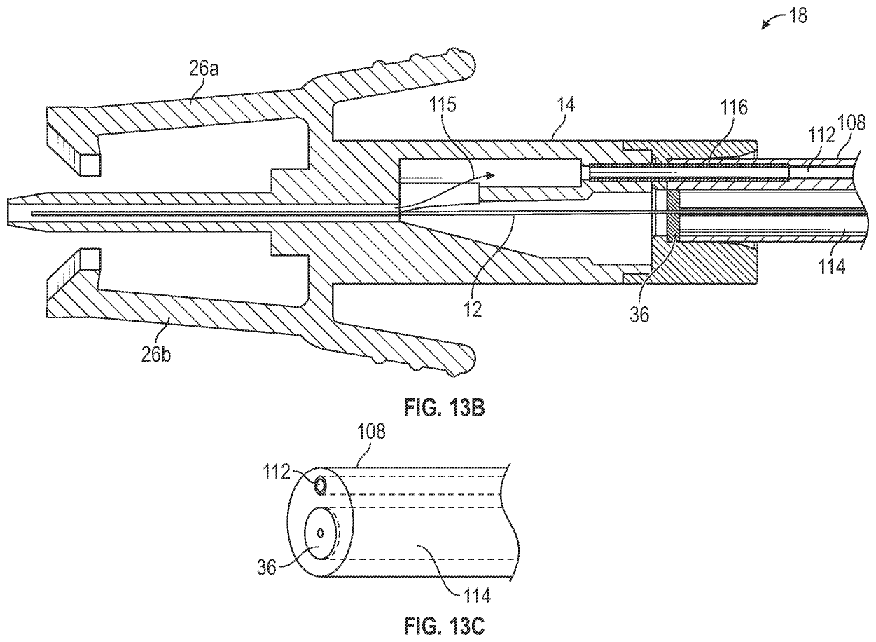

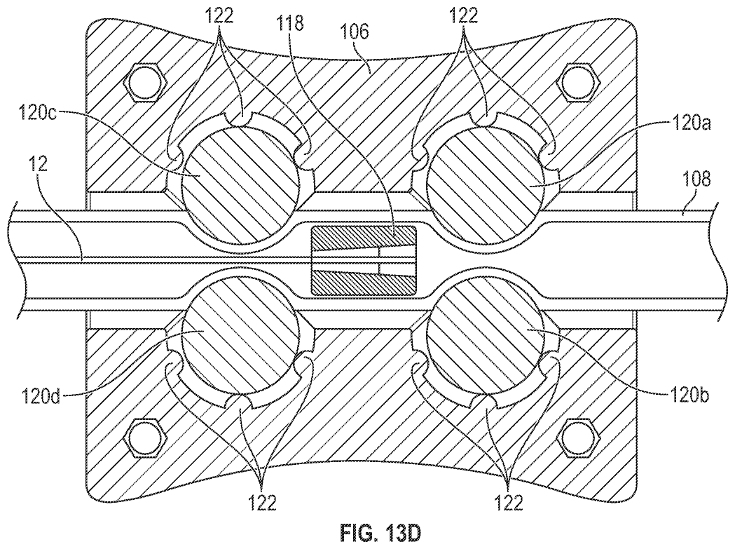

Referring now to FIG. 13A-13C, an instrument advancement device 104 is illustrated, according to some embodiments. In some embodiments, the instrument advancement device 104 may be similar or identical to the instrument advancement device 10 in terms of one or more features and/or operation. In some embodiments, the instrument advancement device 104 may include a housing 106 and an extension tube 108 extending through the housing 106. In some embodiments, the extension tube 108 may be rigid, semi-rigid, or flexible. In some embodiments, the extension tube 108 may include a multi-lumen extension tube.

In some embodiments, the instrument advancement device 104 may include the instrument 12. In these and other embodiments, the instrument 12 may be lubricated or coated to ease advancement. In some embodiments, the instrument 12 may be configured to extend into and/or through the catheter assembly to push through and/or disrupt an occlusion of the catheter.

In some embodiments, a distal end of the instrument advancement device 104 may include a connector 24 or another suitable connector. In some embodiments, the connector 24 may be configured to couple to the catheter assembly, which may be existing or already dwelling within the vasculature of the patient. In some embodiments, the catheter assembly may include an introducer needle, which may extend through the catheter and facilitate piercing of skin and the vasculature to insert the catheter into the patient. In some embodiments, the introducer needle may be removed from the catheter assembly prior to coupling of the instrument advancement device 104 to the catheter assembly.

In some embodiments, a distal end of the extension tube 108 may be coupled to the connector 24. In some embodiments, a proximal end of the extension tube 108 may be coupled to a proximal adapter 110, which may include another distal adapter or another suitable connector. In some embodiments, the proximal adapter 110 may be configured to couple to a blood collection device. In some embodiments, the blood collection device may include a syringe, a BD VACUTAINER® blood collection tube available from Becton Dickinson & Company of Franklin Lakes, N.J., or another suitable blood collection device, which may provide suction.

In some embodiments, the instrument 12 may be advanced prior to or during infusion or blood draw. In some embodiments, after completing a blood draw or infusion and before uncoupling the instrument advancement device 104 from the catheter assembly, the user may retract the instrument by moving the housing 106 backward or proximally. Thus, in some embodiments, a risk of exposure of the user to blood may be decreased.

As illustrated in FIGS. 13B-13C, in some embodiments, the extension tube 108 may include a first lumen 112 and a second lumen 114, which may be separate from the first lumen 112 along an entire length of the extension tube 108. In some embodiments, a blood collection pathway may extend through the first lumen 112. In some embodiments, the instrument 12 may be disposed within the second lumen 114. In some embodiments, a diameter of the second lumen 114 may be larger than a diameter of the first lumen 112. In some embodiments, the diameter and/or a length of the first lumen 112 may be selected based on a desired flow rate and/or to reduce hemolysis.

In some embodiments, in response to moving the housing 106 distally along the extension tube 108, the instrument 12 may be advanced distally within the second lumen 114. In some embodiments, in response to moving the housing 106 proximally along the extension tube 108, the instrument 12 may be retracted proximally within the second lumen 114.

In some embodiments, the instrument advancement device 104 may include a septum 36 disposed within the connector 24 and configured to seal the second lumen 114 or prevent blood flow into the second lumen 114. In these and other embodiments, the septum 36 may not seal the first lumen 112 such that blood may flow proximally along a fluid pathway 115 from the connector 24 through the first lumen 112 for blood collection. In some embodiments, the septum 36 may be elastomeric.

In some embodiments, a distal end of the instrument 12 may be disposed proximal to a distal end of the connector 24 when the housing 106 is fully retracted in a proximal direction. In some embodiments, the distal end of the instrument 12 may be disposed proximal to the septum 36 when the housing 106 is fully retracted in the proximal direction and/or the instrument 12 may be sealed within the extension tube 108.

In some embodiments, the instrument advancement device 104 may include a cannula 116, which may connect a distal end of the first lumen 112 and the connector 24. In some embodiments, the cannula 116 may be blunt. In some embodiments, the fluid pathway 115 may extend through the cannula 116, which may prevent blood leakage. In some embodiments, the cannula 116 may be constructed of steel, plastic, metal, or another suitable material. In some embodiments, the cannula 116 may be coupled to the connector 24 or monolithically formed with the connector 24 as a single unit. In some embodiments, the septum 36 may be concentric with the second lumen 114 or offset slightly to obtain adequate wall thicknesses. In some embodiments, the septum 36 may extend across a width of the second lumen 114. In some embodiments, the septum 36 may seal the second lumen 114 and prevent blood flow into the second lumen 114.

Referring now to FIG. 13D, in some embodiments, the instrument advancement device 104 may include a wedge 118 disposed within the housing 106 and the second lumen 114 of the extension tube 108. In some embodiments, the instrument advancement device 104 may include a pair of opposing pinch members 120 configured to pinch the extension tube 108. In some embodiments, the pair of opposing pinch members 120a,b may be disposed within the housing 106 proximal to the wedge 118 and configured to move along the extension tube 108 with the housing 106.

In some embodiments, the instrument 12 may extend distally from the wedge 118. In some embodiments, the instrument 12 may be disposed within the second lumen 114. In some embodiments, in response to moving the housing 106 distally along the extension tube 108, the pair of opposing pinch members 120a,b may push the wedge 118 distally, and the instrument 12 may be advanced distally.

In some embodiments, the instrument advancement device 104 may include another pair of opposing pinch members 120c,d configured to pinch the extension tube 108. In some embodiments, the other pair of opposing pinch members 120c,d may be disposed within the housing distal to the wedge 118 and configured to move along the extension tube 108 with the housing 106. In some embodiments, in response to moving the housing 106 proximally along the extension tube 108, the pair of opposing pinch members 120c,d may push the wedge 118 proximally and the instrument 12 may be retracted proximally.

The pair of opposing pinch members 120a,b and the other pair of opposing pinch members 120c,d may be referred to collectively in the present disclosure as “opposing pinch members 120.” In some embodiments, in response to movement of the housing 106 along the extension tube 108, the opposing pinch members 120 may rotate with respect to the housing 106 and the extension tube 108. In some embodiments, in response to movement of the housing 106 along the extension tube 108, the opposing pinch members 120 may rotate with respect to the housing 106 and the extension tube 108, which may rotate the instrument 12. In some embodiments, an inner surface of the housing 106 may include one or more bumps 122 in contact with the opposing pinch members 120, which may reduce friction as the opposing pinch members 120 rotate. In some embodiments, the wedge 118 and/or the opposing pinch members 120 may be lubricated with a lubricant, which may reduce friction.

In some embodiments, the opposing pinch members 120 may be constructed of plastic, metal, or another suitable material. In some embodiments, the opposing pinch members 120 may include spherical balls, ball bearings, wheels, or cylinders, which may be configured to rotate with respect to the housing 106. In some embodiments, the opposing pinch members 120 may include the wheels, which may be smooth or include feet along their edges. In these embodiments, lubricant may be applied to axels of the wheels to reduce friction. In some embodiments, the opposing pinch members 120 may be fixed with respect to the housing 106. For example, the opposing pinch members 120 may be molded into the housing 106.

In some embodiments, a number of the opposing pinch members 120 may vary based on a shape of the wedge 118. In some embodiments, the instrument advancement device 104 may include the pair of opposing pinch members 120a,b and the other pair of opposing pinch members 120c,d in response to the shape of the wedge 118 being cylindrical, for example. In some embodiments, the instrument advancement device 104 may include a single pair of the opposing pinch members 120, such as the pair of the opposing pinch members 120a,b, in response to the wedge 118 including a dog bone shape, and the single pair may be disposed in a middle or depression of the dog bone shape.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. An instrument advancement device to advance an instrument into a catheter
assembly, the instrument advancement device comprising:
a housing, comprising a proximal end, a distal end, and a slot;
an advancement element extending through the slot and configured to move linearly along the slot between a retracted position and an advanced position;
a septum disposed within the housing, wherein:
a distal face of the septum is flat; or
a distal face of the septum is bulging and a proximal face of the septum is bulging; or
a distal face of the septum is concave and a proximal face of the septum is concave; and
an instrument comprising a first end and a second end, wherein in response to movement of the advancement element from the retracted position to the advanced position, the instrument moves through the septum and the second end of the instrument is advanced beyond the distal end of the housing.

2. The instrument advancement device of claim 1, wherein the distal face of the septum is flat.

3. The instrument advancement device of claim 1, wherein the distal face of the septum is bulging and the proximal face of the septum is bulging.

4. The instrument advancement device of claim 1, wherein the distal face of the septum is concave and the proximal face of the septum is concave.

5. An instrument advancement device to advance an instrument into a catheter assembly, the instrument advancement device comprising:
a housing, comprising a proximal end, a distal end, a slot, and a support wall, wherein the support wall comprises an opening configured to receive the instrument therethrough;
an advancement element extending through the slot and configured to move linearly along the slot between a retracted position and an advanced position;
a septum disposed within the housing, wherein a distal face of the septum is in contact with the support wall; and
an instrument comprising a first end and a second end, wherein in response to movement of the advancement element from the retracted position to the advanced position, the instrument moves through the septum and the second end of the instrument is advanced beyond the distal end of the housing.

6. The instrument advancement device of claim 5, wherein the support wall extends perpendicular to a longitudinal axis of the housing.

7. The instrument advancement device of claim 5, wherein a center of the septum is aligned with the opening.

8. The instrument advancement device of claim 5, wherein the septum comprises a duckbill valve.

9. The instrument advancement device of claim 5, further comprising another septum, wherein the septum is in contact with a proximal side of the support wall, wherein the other septum is in contact with a distal side of the support wall.

10. The instrument advancement device of claim 5, wherein a portion of the septum aligned with opening comprises a proximally-facing pocket configured to receive the instrument therethrough, further comprising a lube disposed within the proximally-facing pocket.

11. The instrument advancement device of claim 5, further comprising a wedge, wherein the wedge comprises a distal end and an outwardly-extending mouth portion opposite the distal end, wherein the distal end of the wedge is secured within the opening of the support wall, wherein a proximal face of the septum is in contact with a distal side of the support wall, wherein the outwardly-extending mouth portion comprises a lube therein.

12. An instrument advancement device to advance an instrument into a catheter
assembly, the instrument advancement device comprising:
a housing, comprising a proximal end, a distal end, and a slot;
an advancement element extending through the slot and configured to move linearly along the slot between a retracted position and an advanced position;
a septum disposed within the housing;
an instrument comprising a first end and a second end, wherein in response to movement of the advancement element from the retracted position to the advanced position, the instrument moves through the septum and the second end of the instrument is advanced beyond the distal end of the housing;
a lube disposed within the housing and configured to facilitate advancement of the instrument through the septum.

13. The instrument advancement device of claim 12, further comprising another septum disposed within the housing, wherein the lube fills a volume between the septum and the other septum.

14. The instrument advancement device of claim 12, wherein the septum comprises a proximally-facing pocket, further comprising a wedge, wherein a distal end of the wedge is disposed within the proximally-facing pocket of the septum.

15. The instrument advancement device of claim 13, wherein an outwardly-angled mouth portion of the wedge comprises the lube therein.

16. The instrument advancement device of claim 15, wherein the outwardly-angled mouth portion of the wedge is disposed proximal to the septum.

17. The instrument advancement device of claim 12, wherein a distal face of the septum is flat, wherein a proximal face of the septum comprises a pocket configured to receive the instrument therethrough, further comprising lube disposed within the pocket.

18. The instrument advancement device of claim 12, wherein the septum comprises a cavity enclosed by a body of the septum, wherein the lube is disposed within the cavity enclosed by the body of the septum.

19. The instrument advancement device of claim 12, wherein a distal face of the septum comprises a distally-facing pocket, further comprising a film or another septum, wherein the lube is sealed within the distally-facing pocket by the film or the other septum, wherein the lube fills a volume between the septum and the film, in response to movement of the advancement element from the retracted position to the advanced position, the instrument is configured to puncture the film or the other septum to release the lube.

20. The instrument advancement device of claim 12, further comprising a film proximal to the septum, wherein the lube fills a volume between the septum and the film, in response to movement of the advancement element from the retracted position to the advanced position, the instrument is configured to puncture the film to release the lube.
